(19) United States
(12) Patent Application Publication
(10) Pub. No.: US 2005/0229336 A1
(43) Pub. Date: Oct. 20, 2005

(54) Method of treating hair fibers

(75) Inventors: Thomas Fondin, Taverny (FR); Anne Sabbagh, Rueil Malmaison (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL

(21) Appl. No.: 11/097,154

(22) Filed: Apr. 4, 2005

Related U.S. Application Data
(60) Provisional application No. 60/571,896, filed on May 18, 2004.

(30) Foreign Application Priority Data
Apr. 2, 2004 (FR) 04 50666

(51) Int. Cl. A61K 7/13

(52) U.S. Cl. 8/405

(57) ABSTRACT

The present disclosure relates to a method for treating hair fibers comprising: applying to the hair fibers at least one reducing composition comprising at least one reducing agent chosen form non-sulfur containing agents and protected thiols, and then applying a heating iron to the hair fibers to increase the temperature of the hair fibers, wherein the heating iron has a temperature of at least 60° C., and wherein the heating iron is applied before or after the hair fibers are optionally rinsed.

This application claims benefit of U.S. Provisional Application No. 60/571,896 filed May 18, 2004, and French Patent Application No. 04 50666, filed Apr. 2, 2004, both of which are hereby incorporated by reference.

The present disclosure relates to a method of treating hair fibers.

The usual practice to permanently reshape hair comprises first opening the keratin disulfide bonds (cystine) with a composition comprising a reducing agent. The disulfide bonds are then re-formed, optionally after rinsing the hair, by applying to the hair, which has been straightened or placed beforehand under tension with suitable means such as curlers or the equivalent, an oxidizing composition also called a fixing solution, so as to give the desired form to the hair. This method results in the waving of the hair, the uncurling of the hair, the backcombing of the hair, or the straightening of the hair.

Reducing compositions that may be used to carry out the first step of these methods generally comprise some thiol group comprising compounds, such as thioglycolic acid, cysteine, cysteamine, thiolactic acid, and glycerol monothioglycolate.

Nevertheless, such a method may not be fully satisfactory. While it is usually very efficient for reshaping the hair, it may cause great damage to the hair fiber.

Moreover, it has been suggested to increase the temperature of the hair fibers between the reducing step and the fixing step by devices such as a heating iron.

Japanese Patent Application Publication No. JP 2000-256146 describes a process to permanently reshape the hair, comprising the application of a cosmetic composition comprising from 2% to 11% reducing agents and from 0.2% to 4% diammonium dithioglycolate. After the reducing composition is applied, a heating iron is used at a temperature from 60° C. to 220° C.

Nevertheless, using a heating iron may cause the resulting shape to be irreversible. Moreover, there are noticeable differences between the treated hair fibers and the roots of the hair fibers as the hair regrows.

Finally, if the treatment is carried out on colored hair, then it may very frequently cause the hair color to fade as a result of the treatment.

It would thus be desirable to provide a hair fiber treating method that compensates for the drawbacks of the prior art.

In one aspect, the present disclosure provides for a hair fiber treating method enabling a change in hair fiber behavior while limiting the damage caused to the hair fiber, enabling control of the hair volume, and/or enhancing at least one cosmetic benefit provided to the hair, such as softness, shine and ease of combing, and may simultaneously also better preserve colored hair shades.

The method may also be able to preserve the hair's natural aspects so as to limit the so-called ‘root effect’, that is to say the difference between the hair fibers which have been treated and the roots.

In a further embodiment, the method of the present disclosure may reduce the hair fiber treating time and give long-lasting results.

For example, the present inventors have discovered that the method disclosed herein may counteract at least one of the drawbacks of the prior art and achieve at least one of the desirable results listed above. This method comprises applying onto the hair fibers a reducing composition comprising at least one particular reducing agent, then applying a heating iron to the hair fibers to increase their temperature, wherein the heating iron has a temperature of at least 60° C., and wherein the temperature increasing step may be conducted before or after an optional hair fiber rinsing step.

More specifically, the present disclosure provides a method for treating hair fibers comprising:
applying onto the hair fibers a reducing composition comprising at least one reducing agent chosen from non-sulfur and protected thiol reducing agents; and
applying a heating iron to the hair fibers to increase the temperature of the hair fibers, the heating iron having a temperature of at least 60° C.,
wherein the heating iron is applied before or after an optional rinsing of the hair fibers.

As used herein, “protected thiol” means any molecule that may generate a thiol group upon a chemical or photochemical simple reaction (e.g., a hydrolysis reaction).

In one embodiment, the at least one reducing agent is a non-sulfur reducing agent. As used herein, “non-sulfur reducing agents” mean a reducing agent the structure of which does not comprise any sulfur atoms. In another embodiment, the present disclosure provides for a method for treating hair fibers comprising:
applying onto the hair fibers a reducing composition free of ceramide and comprising at least one reducing agent chosen from sulfites and bisulfites; and
applying a heating iron to the hair fibers to increase the temperature of the hair fibers, wherein the heating iron has a temperature of at least 60° C., and wherein the heating iron is applied either before or after an optional rinsing of the hair fibers.

In one embodiment, the reducing composition may be applied onto wet and clean hair fibers.

In one embodiment, the non-sulfur reducing agent in the composition according to the present disclosure is, for example, chosen from phosphines, and alkali metal and alkaline-earth metal borohydrides.

Sulfites and bisulfites that are suitable for use in the composition according to the present disclosure may be, for example, chosen from alkali metal, alkaline-earth metal, and ammonium sulfites and bisulfites, and further for example, sodium and potassium sulfite and bisulfite, alkanolamine sulfites and bisulfites such as monoethanolamine sulfite and bisulfite.

Sodium and potassium borohydrides are, for example, suitable for use in the composition according to the present disclosure.

Among the protected thiol reducing agents suitable for use herein, mention may be made of, for example, thioesters, thiocarbonates, thiocarbamates, and thioethers.

When the reducing composition comprises at least one sulfite or bisulfite reducing agent, the reducing composition may be free from dithiodiglycolic acid or any salt thereof.

The at least one reducing agent is present in an amount ranging from 0.1% to 30%, such as from 0.5% to 20%, and further for example, from 1% to 10% by weight, as compared to the total weight of the reducing composition.

The reducing composition may have a pH value ranging from 2 to 13, such as from 6 to 10.

The reducing composition pH may be balanced by an agent chosen from alkaline agents such as, for example, ammonia; organic amines such as monoethanolamine, diethanolamine, triethanolamine, 1,3-propanediamine and 2-amino-2-methyl-1-propanol; alkaline and ammonium carbonate and bicarbonate; organic carbonate such as guanidine carbonate; alkaline hydroxide such as soda; and acidifying agents such as, for example, hydrochloric acid, acetic acid, lactic acid, oxalic acid, and boric acid.

The reducing composition may comprise at least one cosmetically acceptable solvent chosen from water; C₁-C₆ alcohols, for example, alkanols such as ethanol, propanol, and isopropanol; polyhydric alcohols, such as propyleneglycol, pentanediol and glycerine; benzyl alcohol; polyol ethers; C₂-C₆ esters; N-methylpyrrolidone (NMP); and C₃-C₆ cetones.

In order to improve the cosmetic properties of the hair fibers or to reduce or prevent their damaging, the reducing composition as used according to the present disclosure may further comprise at least one cosmetic additive.

The at least one cosmetic additive is chosen from volatile and non volatile, linear and cyclic silicones; cationic polymers; peptides and derivatives thereof; protein hydrolyzates; waxes; swelling agents and penetrating agents; agents that are capable of increasing the efficiency of the at least one reducing agent such as a SiO₂/polydimethylsiloxane mixture; dimethylisosorbitol; urea and derivatives thereof; anionic, cationic, non ionic, amphoteric and zwitterionic surfactants; active agents capable of combating hair loss; anti-dandruff agents; natural and synthetic, associative and unassociative thickeners; suspension agents; sequestering agents; opacifying agents; dyes; sunscreen agents; vitamins and provitamins; mineral, vegetable and synthetic oils; fragrances and preserving agents; and any combination thereof.

The reducing composition used in the method according to the present disclosure may be in a form chosen from an optionally thickened lotion; a cream; a gel; and a foam.

The method disclosed herein comprises applying the at least one reducing composition as defined above to hair fibers. Once the at least one reducing composition has been applied, it can be left on the hair fibers, optionally under a drying helmet, for a time period ranging from 5 to 60 minutes, for example, from 5 to 30 minutes.

As explained above, the method according to the disclosure comprises following the reducing composition application step, with an optional rinsing step, then increasing the temperature of the hair fibers by applying a heating iron at a temperature of at least 60° C.

As used herein, “heating iron” means a heating device which functions by contacting the hair fibers.

The end of the iron coming into contact with the hair may have various forms. For example, the heating iron may have a plane surface, which defines a so-called flat iron. The heating iron may also have a rounded surface, which then defines a round iron.

The iron may be applied proceeding by successive separated touches over about a few seconds or by gradually moving or sliding along hair locks.

All types of flat or round irons may be given as non-limiting examples of suitable irons for use in the method according to the disclosure, and for example, those described in U.S. Pat. Nos. 4,103,145, 4,308,878, 5,983,903, 5,957,140, 5,494,058, and 5,046,516.

The hair fiber temperature may be increased to a temperature ranging from 60° C. to 250° C., such as from 120° C. to 220° C.

According to one embodiment, the hair fibers are not rinsed out before increasing their temperature with a heating iron.

The method according to the present disclosure may further comprise another step comprising partially pre-drying the hair fibers before applying the heating iron, so as to prevent any substantial steam development that might burn the hands of the hair stylist and the scalp of the user. The pre-drying step may be done, for example, by using a drying device such as a hair drier, a hood or it is also possible to let the hair dry naturally.

When the at least one reducing composition comprises non-sulfur or protected thiol reducing agents, the method according to the disclosure may further comprise after increasing the temperature of the hair fibers, fixing the hair fibers by applying an oxidizing composition.

In one embodiment, the oxidizing step, i.e., applying the oxidizing composition, is only conducted when the at least one reducing agent is a non-sulfur reducing agent-based reducing composition.

The oxidizing composition used for the fixing step may comprise at least one oxidizing agent chosen from hydrogen peroxide; urea peroxide; alkali metal bromates; persalts, such as perborates and persulfates; and enzymes, such as peroxidases and two-electrons oxidoreductases.

In one embodiment, the hydrogen peroxide may be present in a concentration ranging from 1 to 10 volumes and for example, from 6 to 8 volumes.

In another embodiment, bromate may be present in an a concentration ranging from 1% to 12%, by weight and persalt may be present in a concentration ranging from 0.1% to 15% by weight, as compared to the total weight of the oxidizing composition.

When the method according to the disclosure comprises a fixing step, the method may be, for example, carried out by using a cosmetic kit, comprising, in a first compartment, a reducing composition such as defined above and, in a second compartment, an oxidizing composition such as defined above.

In one embodiment, the method according to the disclosure does not comprise any fixing step comprising applying an oxidizing composition.

Finally, the present disclosure also relates to a method for durably changing hair shape without excessively altering the hair color and/or without excessively damaging the hair fibers.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The disclosure may be understood more clearly with the aid of the non-limiting examples that follow.

### EXAMPLES

The hair fiber treating method according to the disclosure was carried out using a reducing composition.

Tested reducing compositions were as follows:

| | |
|---|---|
| Reducing composition 1 | |
| Trihydroxyethyl phosphine | 1.5 g |
| MEXOMERE PO | 2.5 g |
| pH agent | qs pH 9 |
| Demineralized water | qs 100 g |
| Reducing composition 2 | |
| Ethanolamine sulfite | 1.5 g |
| MEXOMERE PO | 2.5 g |
| pH agent | qs pH 7 |
| Demineralized water | qs 100 g |
| Reducing composition 3 | |
| Succinimidylacetyl thiacetate | 1.5 g |
| MEXOMERE PO | 2.5 g |
| pH agent | qs pH 9 |
| Demineralized water | qs 100 g |

Tests were conducted on colored, naturally curling hair.

A reducing composition such as previously described was applied onto the hair and left on for about 5 minutes.

The hair was then partially pre-dried using a hair drier before being treated using a flat iron heated to 180° C.

As a result, the hair fiber showed a good texture, a well controlled volume, a good respect of the color, and a long term durability.

1. A method for treating hair fibers comprising:
applying to the hair fibers at least one reducing composition comprising at least one reducing agent chosen from non-sulfur containing reducing agents and protected thiols, and then
applying a heating iron to the hair fibers to increase the temperature of the hair fibers, wherein the heating iron has a temperature of at least 60° C., and wherein the heating iron is applied before or after the hair fibers are optionally rinsed.

2. A method for treating hair fibers comprising:
applying to the hair fibers at least one reducing composition free from ceramide and comprising at least one reducing agent chosen from sulfites and bisulfites, and then
applying a heating iron to the hair fibers to increase the temperature of the hair fibers, wherein the heating iron has a temperature of at least 60° C., and wherein the heating iron is applied before or after the hair fibers are optionally rinsed.

3. The method according to claim 2, wherein the reducing composition is free from dithiodiglycolic acid or any salt thereof.

4. The method according to claim 1, wherein the heating iron has a temperature of from 60° C. to 250° C.

5. The method according to claim 4, wherein the heating iron has a temperature of from 120° C. to 220° C.

6. The method according to claim 2, wherein the heating iron has a temperature of from 60° C. to 250° C.

7. The method according to claim 6, wherein the heating iron has a temperature of from 120° C. to 220° C.

8. The method according to claim 1, wherein the at least one reducing composition is applied onto wet and clean hair fibers.

9. The method according to claim 2, wherein the at least one reducing composition is applied onto wet and clean hair fibers

10. The method according to claim 1, further comprising, after applying onto the hair fibers the at least one reducing composition, leaving it on the hair fibers for a period of time before applying the heating iron.

11. The method according to claim 2, further comprising, after applying onto the hair fibers the at least one reducing composition, leaving it on the hair fibers for a period of time before applying the heating iron.

12. The method according to claim 1, wherein the hair fibers are not rinsed out before the heating iron is applied.

13. The method according to claim 2, wherein the hair fibers are not rinsed out before the heating iron is applied.

14. The method according to claim 1, further comprising partially pre-drying the hair fibers before applying the heating iron.

15. The method according to claim 2, further comprising partially pre-drying the hair fibers before applying the heating iron.

16. The method according to claim 1, wherein the non-sulfur reducing agent is chosen from phosphines, and alkali metal and alkaline-earth metal borohydrides.

17. The method according to claim 1, wherein the protected thiol reducing agent is chosen from thioesters, thiocarbonates, thiocarbamates, and thioethers.

18. The method according to claim 2, wherein the sulfite and/or bisulfite reducing agents are chosen from alkali metal and alkaline-earth metal, ammonium and alkanolamine sulfites and/or bisulfites.

19. The method according to claim 1, wherein the at least one reducing agent is present in an amount ranging from 0.1% to 30%, relative to the total weight of the reducing composition.

20. The method according to claim 19, wherein the at least one reducing agent is present in an amount ranging from 1% to 10%, relative to the total weight of the reducing composition.

21. The method according to claim 2, wherein the at least one reducing agent is present in an amount ranging from 0.1% to 30%, relative to the total weight of the reducing composition.

22. The method according to claim 21, wherein the at least one reducing agent is present in an amount ranging from 1% to 10%, relative to the total weight of the reducing composition.

23. The method according to claim 1, wherein the at least one reducing composition has a pH value from 2 to 13.

24. The method according to claim 23, wherein the at least one reducing composition has a pH value from 6 to 10.

25. The method according to claim 2, wherein the at least one reducing composition has a pH value from 2 to 13.

26. The method according to claim 25, wherein the at least one reducing composition has a pH value from 6 to 10.

27. The method according to claim 1, wherein the at least one reducing composition further comprises at least one cosmetically acceptable solvent chosen from water, C₁-C₆ alcohols, polyhydric alcohols, benzyl alcohol, polyol ethers, C₂-C₆ esters, N-methylpyrrolidone (NMP), and C₃-C₆ cetones.

28. The method according to claim 2, wherein the at least one reducing composition further comprises at least one cosmetically acceptable solvent chosen from water, C₁-C₆ alcohols, polyhydric alcohols, benzyl alcohol, polyol ethers, C₂-C₆ esters, N-methylpyrrolidone (NMP), and C₃-C₆ cetones.

29. The method according to claim 1, wherein the reducing composition further comprises at least one cosmetically acceptable additive chosen from volatile and non volatile, linear and cyclic silicones; cationic polymers; peptides and derivatives thereof; protein hydrolyzates; waxes; swelling agents and penetrating agents; agents capable of increasing the efficiency of the at least one reducing agent; dimethylisosorbitol; urea and derivatives thereof; anionic, cationic, non ionic, amphoteric and zwitterionic surfactants; active agents capable of combating hair loss; anti-dandruff agents; natural and synthetic, associative and unassociative thickeners; suspension agents; sequestering agents; opacifying agents; dyes; sunscreen agents; vitamins and provitamins; mineral, vegetable and synthetic oils; fragrances and preserving agents; and any combination thereof.

30. The method according to claim 1, wherein the reducing composition is in a form chosen from an optionally thickened lotion, a cream, a gel, and a foam.

31. The method according to claim 2, wherein the reducing composition further comprises at least one cosmetically acceptable additive chosen from volatile and non volatile, linear and cyclic silicones; cationic polymers; peptides and derivatives thereof; protein hydrolyzates; waxes; swelling agents and penetrating agents; agents capable of increasing the efficiency of the at least one reducing agent; dimethylisosorbitol; urea and derivatives thereof; anionic, cationic, non ionic, amphoteric and zwitterionic surfactants; active agents capable of combating hair loss; anti-dandruff agents; natural and synthetic, associative and unassociative thickeners; suspension agents; sequestering agents; opacifying agents; dyes; sunscreen agents; vitamins and provitamins; mineral, vegetable and synthetic oils; fragrances and preserving agents; and any combination thereof.

32. The method according to claim 2, wherein the reducing composition is in a form chosen from an optionally thickened lotion, a cream, a gel, and a foam.

33. The method according to claim 1, wherein when the at least one reducing agent is a non-sulfur and/or protected thiol reducing agent, the method further comprises applying an oxidizing composition following applying the heating iron to the hair fibers.

34. The method according to claim 33, wherein the oxidizing composition comprises at least one oxidizing agent chosen from hydrogen peroxide, urea peroxide, alkali metal bromates, persalts, and enzymes.

35. A method for treating hair fibers to change the shape of the hair fibers without excessively altering the hair color and/or without excessively damaging the hair fibers, said method comprising:
applying to the hair fibers at least one reducing composition comprising at least one reducing agent chosen from non-sulfur containing reducing agents and protected thiols, and then
applying a heating iron to the hair fibers to increase the temperature of the hair fibers, wherein the heating iron has a temperature of at least 60° C., and wherein the heating iron is applied before or after the hair fibers are optionally rinsed

36. A cosmetic kit comprising, in a first compartment, a reducing composition comprising at least one reducing agent chosen from non-sulfur and protected thiol reducing agents, and, in a second compartment, an oxidizing composition.
